(19) United States
(12) Patent Application Publication
(10) Pub. No.: US 2015/0327807 A1
(43) Pub. Date: Nov. 19, 2015

(54) DEVICE AND METHOD FOR MONITORING COMPRESSIONS AT A CARDIAC MASSAGE

(71) Applicant: Karl Kuefner GmbH & Co. KG, Albstadt-Truchtelfingen (DE)

(72) Inventors: Rolf BRONNER, Baden-Baden (DE); Joachim HILLER, Goeppingen (DE); Till KAZ, Stuttgart (DE)

(73) Assignee: Karl Kuefner GmbH & Co. KG, Albstadt-Truchtelfingen (DE)

(21) Appl. No.: 14/709,645

(22) Filed: May 12, 2015

(30) Foreign Application Priority Data
May 13, 2014 (DE) 10 2014 209 053.9
Nov. 12, 2014 (DE) 10 2014 116 500.4

(51) Int. Cl. A61B 5/00; A61B 5/087; A61M 16/00; A61M 16/06; A61M 16/08; A61B 5/091; A61B 5/083; A61B 5/097

(52) U.S. Cl. CPC A61B 5/4836; A61B 5/0833; A61B 5/0836; A61B 5/087; A61B 5/097; A61B 5/486; A61B 5/7445; A61B 5/7405; A61M 16/06; A61M 16/0875; A61M 16/085; A61B 5/091; A61B 5/7475; A61B 5/747; A61M 16/0003; A61B 5/6823; A61B 5/6824; A61B 2562/0219; A61B 2560/0214; A61B 2560/0475; A61B 2562/08

(57) ABSTRACT

The invention relates to a device for and method of monitoring compressions in a cardiac massage on a person in a medical emergency situation. The device is equipped with a flow tube (2, 202), which exhibits a continuous flow channel for air, with at least one flow sensor (10) for determining mass or volumetric flow arranged in the flow channel of the flow tube (2, 202), and/or with a vital signs sensor arranged on the flow tube, by means of which a proportion of oxygen and carbon dioxide can be determined, with a processor that produces a characteristic output signal for penetration depth from the mass or volumetric flow determined and/or from the proportion of oxygen or carbon dioxide detected, and

with an output device (124, 224) that issues the output signal.

The invention derives from a device for and a method of monitoring compressions in a cardiac massage.

In an accident or other medical emergency, the affected persons need to be given first aid by first responders until professional rescue services arrive. First responders are individuals who, by chance, are in a position to take life-saving emergency action in an accident or medical emergency. If the person who is affected by a medical emergency has entirely or partially ceased to breathe independently or has suffered a circulatory arrest, measures to resuscitate them and to overcome the respiratory arrest must be taken. These include cardiopulmonary resuscitation. If the airways are blocked or obstructed, the airways of the affected person must first be cleared. It may be necessary for a helper to give the person respiration. In a circulatory arrest a cardiac massage must be performed. The purpose of this measure is to supply the vital organs of the affected person with oxygen.

In the following, the person who is receiving emergency medical attention is described as the person. The person who is providing first aid is described as the helper in the following. In the following, resuscitation measures are those that are suitable for maintaining a circulation to a certain extent in order to supply the most important organs until professional rescue services arrive.

In a cardiac massage, the thorax of the person is compressed by applying pressure. After the pressure is released, decompression takes place. The thorax expands again. This process is constantly repeated. When the thorax is compressed, the person's heart and lungs in particular are compressed. To a limited extent this supports a circulation of the blood to vital organs, in particular the person's brain.

Potential first responders often have no or inadequate knowledge of first aid. For fear of making mistakes due to this inadequate knowledge, often no or inadequate first aid is provided. In addition, potential first responders often do not recognize a respiratory arrest or a circulatory arrest. The necessary assistance is consequently not given.

The invention is based on the task of providing a device for and a method of monitoring compressions in a cardiac massage to facilitate a helper to perform a cardiac massage on a person in a medical emergency, even if the helper has no or inadequate knowledge of first aid.

This task is solved by a device with the characteristics of claim 1 and by a method with the characteristics of claim 22. The device according to the invention is characterized by a flow tube to which a respiratory mask can be connected in such a way that air flows out of the respiratory mask into the flow tube and air flows out of the flow tube into the respiratory mask. The respiratory mask can be fitted over the nose and mouth of a casualty or person in an emergency situation. This may be a known type of respiratory mask. The design of the flow tube at a first end is such that the respiratory mask can be connected to the first end. This is achieved by fitting the respiratory mask into the matching first end of the flow tube with a connecting piece. The respiratory mask is preferably arranged on a casualty or person in an emergency situation in such a way that it remains on the nose and mouth without constantly needing to be held by a helper. The flow tube exhibits a continuous flow channel for air. This air is fed to the person and or flows out of him/her. A flow sensor and/or a vital signs sensor is arranged on the flow tube.

The air led through the flow channel passes around the flow sensor, which determines the mass or volumetric flow of the air flowing through the flow channel. The volumetric flow of a gas flowing through the flow tube here corresponds to a volume that passes through a specified cross-section of the flow tube per unit of time. The mass flow or mass flow rate of a gas flowing through the flow tube corresponds to a mass that passes through a specified cross-section of the flow tube per unit of time.

Compression of the lungs in the cardiac massage results in air flowing out of the nose or mouth of the person, provided the person's airways are not obstructed. This mass or volumetric flow is determined by the flow sensor.

The volumetric flow or flow rate of the breathed air fed to the person is also referred to as the inspiratory flow. The volumetric flow or flow rate of the air flowing out of the person is also referred to as the expiratory flow.

A rate of change in the mass or volumetric flow can also be determined from the mass or volumetric flow.

The vital signs sensor determines the proportion of oxygen and carbon dioxide in the air flowing through the flow channel. The oxygen concentration or oxygen content can be determined from the proportion of oxygen in the air flowing. The carbon dioxide concentration or carbon dioxide can be determined from the proportion of carbon dioxide.

Oxygen is converted into carbon dioxide in the person's lungs. This conversion also occurs during respiration or a cardiac massage if the person is not breathing independently and has suffered circulatory arrest. Therefore, if the sensor demonstrates that the carbon dioxide concentration in the air flowing out of the person is greater than the air fed to the person, this is an indication that air has reached the person's lungs as a result of the cardiac massage.

The respiratory mask rests on the person's face. If required it may be secured to the person's head with a strap or belt to keep it in position. Thanks to its soft, elastic edge, the respiratory mask lies basically airtight on the person's face. This results in most of the air flowing out of the person's nose or mouth reaching the flow tube and flowing through it. In doing so, it passes the flow sensor, so that the mass or volumetric flow of the flowing air is determined.

By means of the device it can be determined whether the casualty or person in an emergency situation has entirely or partially ceased to breathe before a cardiac massage is performed. If the person has ceased to breathe, the flow sensor will indicate no mass or volumetric flow. On the other hand if a mass or volumetric flow is determined, this is a sign that the person is breathing spontaneously.

The vital signs sensor likewise provides an indication of whether a person has ceased to breathe entirely or partially, before a cardiac massage is performed. If there is no spontaneous breathing, the vital signs sensor will indicate no increase in the proportion of carbon dioxide.

If no mass or volumetric flow from the person and/or no change in the proportion of oxygen and carbon dioxide is indicated in a cardiac massage during compression of the person's thorax, this is a sign that the person's airways are obstructed. This applies in particular if no mass or volumetric flow and/or no change in the proportion of oxygen and carbon dioxide is determined despite an increase in penetration depth in the cardiac massage. The airways may be obstructed by the person's tongue or by vomit.

If the airways are clear, it can be ascertained from the mass or volumetric flow determined with the flow sensor and/or from the proportion of oxygen and carbon dioxide whether the penetration depth in the cardiac massage is sufficient to maintain an essential circulation for resuscitation and to supply certain organs. Certain ranges are specified here. The penetration depth corresponds to the distance by which the thorax is pressed in during compression. The mass or volumetric flow and the proportion of oxygen and carbon dioxide shown during compression of the thorax are relative to the penetration depth of the thorax. Within a certain range, the greater the penetration depth in the cardiac massage, the greater the mass or volumetric flow of the air flowing out of the person. Moreover within a certain range, the greater the penetration depth in the cardiac massage, the greater the rise in the proportion of carbon dioxide.

In this connection, resuscitation means that until professional rescue services arrive, a certain circulation can be maintained in the person to supply the most important organs, in particular the brain.

The device is equipped with a processor and an output device. The mass or volumetric flow determined with the flow sensor and/or the proportion of oxygen and carbon dioxide determined with the vital signs sensor is processed in the processor. The signals of the flow sensor and vital signs sensor are processed to that end. An output signal that is output by the output device is generated.

A helper thus receives assistance in administering first aid. The first aid can be adapted optimally to the needs of the person. In this way, inhibitions in potential helpers can be overcome.

The determination of the parameters for the gases flowing in the flow tube also continues during first aid. As a result, the instructions given to the helper during the course of first aid can be adapted continually to any changes in the condition of the person in an emergency situation.

It is beneficial that the device features a data storage device. Data, signals and/or measured values for the mass or volumetric flow determined and/or the proportion of oxygen and carbon dioxide or parameters derived therefrom are stored in the data storage device. In addition, different ranges for a sufficient mass or volumetric flow for resuscitation can be stored in the data storage device for different groups of persons, for example adults, children and infants. Furthermore, different ranges for a sufficient proportion of oxygen and carbon dioxide for resuscitation can be stored for the above groups of persons. In addition to the possible measured values or measured-value ranges, recommendations for the helper on how to proceed can be stored in the data storage device. In this case the processor matches up the measured values determined by the sensor with appropriate instructions or recommendations stored in the data storage device. These are output to the helper by means of an output device. In this way, the helper can be informed continuously of what steps they should take, adapting the steps to the prevailing condition of the casualty or person in an emergency situation. The helper is for example informed when they should start to administer cardiac massage, how often and at what time intervals they should exert pressure to the thorax of the casualty or person in an emergency situation, and whether they should additionally supply breathing air to the casualty or person in an emergency situation.

According to an advantageous embodiment of the invention, additional sensors are provided on the flow tube that determine additional parameters for the air flowing in the flow channel and/or the gases contained in the flowing air. The parameters represent in particular physical or chemical properties of the gases. These include for example:
airway pressure relative to the surroundings,
ambient pressure,
pressure difference,
temperature,
humidity.

The additional sensor or sensors are designed to determine the above parameters. The parameters such as respiratory flow, pressure difference, oxygen or carbon dioxide concentration are also referred to as vital signs.

According to an advantageous embodiment of the invention, the output device is an optical display device. The optical display device exhibits for example a screen or a monitor.

According to a further advantageous embodiment of the invention, the optical display device exhibits a combination of several light sources. Suitable light sources include light-emitting diodes in different colors, for example. The different colors can represent different ranges of the mass or volumetric flow for the person measured with the flow sensor and/or of the proportion of oxygen and carbon dioxide determined with the vital signs sensor or a parameter derived therefrom. For example, a first color can represent a mass or volumetric flow and/or a proportion of oxygen and carbon dioxide that is less than or equal to a specified minimum. A second color can represent a mass or volumetric flow and/or a proportion of oxygen and carbon dioxide that is greater than or equal to a specified maximum. A third color can represent a mass or volumetric flow and/or a proportion of oxygen and carbon dioxide that is between the minimum and the maximum. Different minimum and maximum values can be specified here for different groups of people, for example adults, children and infants. These are preferably stored in a memory device in the device.

According to a further advantageous embodiment of the invention, the optical display device exhibits an optically active surface on which the output signal is displayed. It is beneficial that this optically active surface is convex.

According to a further advantageous embodiment of the invention, the optical display device is a screen.

Alternatively, the surface can also take the form of an essentially flat surface. It is beneficial that the flat optically active surface is aligned at an angle of more than 0° and less than 90° to the longitudinal direction of the flow tube. The longitudinal direction of the flow tube here corresponds essentially to the direction of flow of the gases flowing through the flow tube. The curvature of the optically active surface or the angle of the optically active surface to the flow tube makes it easier for the helper to identify the output signal displayed on the display device while the device is fitted to the person in an emergency situation.

According to a further advantageous embodiment of the invention, the output device exhibits an acoustic output device. In this case the output signal is output by a microphone to the helper, for example. The optical display device and acoustic output display device can also be combined with each other.

According to a further advantageous embodiment of the invention, the output device comprises an interface for the output of data concerning a mass or volumetric flow determined by the flow sensor, or data derived therefrom, to a mobile communications device, in particular a telecommunications device. It can also be an interface for data transmission by radio, such as Bluetooth, or an interface for data transmission over a data line. In this case the output signal can also be output via the mobile telecommunications device. In addition, the output signals can be stored in a memory device of the telecommunications device for retrieval at a later point in time.

Several different output devices can also be provided, so that the steps to be taken are output to the helper in various different ways.

According to a further advantageous embodiment, the device is equipped with an interface over which data concerning a mass or volumetric flow determined by the flow sensor and/or a proportion of oxygen and carbon dioxide or data derived therefrom can be output to an external device for emergency care, to a medical device or to a computer. The data is preferably stored in a data storage device. It is output from the data storage device of the device, over the interface, to an external device. The data can now be called up and is available for the subsequent care of the patient. A paramedic, a doctor or a nurse can now swiftly and easily form an impression of the condition of the person and of what first aid has already been administered. The medical device may for example be a respirator or a defibrillator. The device according to the invention is in particular also suitable for controlling a defibrillator.

According to a further advantageous embodiment of the invention, a first filter is arranged at a second end of the flow tube or between this end and the flow sensor or vital signs sensor in the flow channel. It promotes the flow of the air or the air mixture through the flow tube, along the flow sensor and/or vital signs sensor with the goal of generating an ideally laminar flow. Furthermore, the first filter prevents the penetration of particles into the flow tube that could damage the flow sensor and/or the vital signs sensor. The first filter can for example consist of a plastic or metal lattice or mesh, in particular wire, of a nonwoven material or of a metal and/or plastic fabric.

According to a further advantageous embodiment of the invention, the flow tube is equipped with a connection to which a mouthpiece for the respiration of a person in an emergency situation can be connected. Air can be fed to the person by a helper via this mouthpiece. The breathing air of the helper or ambient air from a pump, for example, can be fed via the mouthpiece. The mouthpiece can for example be connected to a second end of the flow tube. The air fed via the mouthpiece by a helper passes through the flow tube into the respiratory mask, and from there into the person's nose and/or mouth. The mass or volumetric flow of the fed air is determined by the flow sensor when it flows through the flow tube.

According to a further advantageous embodiment of the invention, a second filter is arranged in the flow channel between the first end of the flow tube and the flow sensor or vital signs sensor in the flow channel. The second filter ensures that the air entering through the filter flows as evenly as possible around the flow sensor and/or the vital signs sensor. The aim is to generate an ideally laminar flow. In addition, the second filter prevents particles from reaching the flow tube and damaging the flow sensor and/or the vital signs sensor. These include emissions by the person in a medical emergency situation. The second filter can for example consist of a plastic or metal lattice or mesh, in particular wire, of a nonwoven material or of a metal and/or plastic fabric.

According to a further advantageous embodiment of the invention, a third filter is arranged between the respiratory mask and the flow sensor and/or the vital signs sensor to trap moisture in the air flowing out of the person, thus preventing the person's lungs from drying out excessively. This third filter can in addition trap emissions by the person. The third filter can for example consist of a plastic or metal lattice or mesh, in particular wire, of a nonwoven material or of a metal and/or plastic fabric.

According to a further advantageous embodiment of the invention, the first filter and the second filter are arranged on the flow tube.

According to a further advantageous embodiment of the invention, the cross-section of the second end of the flow tube is matched to known respirators and their accessories. Upon the arrival of professional rescue services, they can connect a respirator to the flow tube. The respiratory mask and the flow tube can remain on the patient.

According to a further advantageous embodiment of the invention, a sensor fixture device is arranged on one wall of the flow tube. The sensor fixture device extends at least partly into the flow channel of the flow tube. The flow sensor and/or the vital signs sensor is arranged on the section of the sensor fixture device that extends into the flow channel. In this way, the flow sensor and/or the vital signs sensor is secured to the flow tube in such a way that it extends into the gas flowing in the flow tube and reliably determines the mass and volumetric flow.

According to a further advantageous embodiment of the invention, the device is equipped with a push-on part in which the processor and the output device are arranged. Optionally, a data storage device and one or more interfaces, as appropriate, are additionally arranged in the push-on part. The push-on part is connected detachably to the flow tube. It can be separated from the flow tube without the push-on part or the flow tube becoming damaged or destroyed. The push-on part exhibits a housing in which the processor and the output device are arranged in a well-protected manner. An energy storage device can in addition be arranged in the push-on part. Because the push-on part does not come into contact with the patient and the breathing air, it can be reused. The flow tube, the mouthpiece and the respiratory mask can either be disposed of after use or cleaned, disinfected and reused.

According to a further advantageous embodiment of the invention, the push-on part encompasses the flow tube at least in part.

According to a further advantageous embodiment of the invention, the push-on part can be laid down on the thorax of a person or fastened to the arm of a helper. This involves preferably detaching the push-on part from the flow tube.

Furthermore, the push-on part is equipped with an acceleration sensor that determines acceleration in the thorax of the person or in the arm of the helper administering cardiac massage. If the flow sensor and/or the vital signs sensor determines that the airways of the person are obstructed, and if it should not be possible to clear the airways, the helper can detach the push-on part from the flow tube and fasten it to his/her arm or lay it on the thorax of the person. For administering cardiac massage, the penetration depth is now determined from the acceleration with which the helper moves his/her arm or the thorax of the person during cardiac massage, instead of by determining the mass or volumetric flow or the proportion of oxygen and carbon dioxide. Changeover from the flow sensor or the vital signs sensor to the acceleration sensor takes place preferably automatically when the push-on part is detached from the flow tube. Here, the flow sensor and the vital signs sensor remain on the flow tube.

According to a further advantageous embodiment of the invention, the processor is designed such that it generates an output signal that can be output with the output device from the acceleration detected with the acceleration sensor.

According to a further advantageous embodiment of the invention, at least two grip recesses and/or outward-protruding handle elements are arranged on the push-on part.

According to a further advantageous embodiment of the invention, the push-on part is equipped with an arm bangle with which it can be fastened to the arm of a helper.

According to a further advantageous embodiment of the invention, the sensor takes the form of a respiration response sensor that determines the respiration response characteristic of the person. Furthermore the processor takes the form of a respiration response processor that evaluates the respiration characteristic and determines whether the airways of the person are obstructed. The result of the evaluation is output via the output device. In this way a helper obtains information on whether the airways of the person to be given respiration are obstructed or clear.

According to a further advantageous embodiment of the invention, the device exhibits an input device via which certain characteristics of the person may be input. Thus, the helper can for example select at the input device whether the person is an adult, a child or an infant. The ranges specified for resuscitation differ between these groups of people, with the result that the helper can be informed accordingly.

According to a further advantageous embodiment of the invention, the processor is designed such that it generates a respiration output signal if the mass or volumetric flow determined and/or the proportion of oxygen or carbon dioxide determined passes above or below a specified threshold value for respiration. Furthermore the output device is designed such that it outputs the respiration output signal. The compressions can be detected and counted by the flow sensor or the vital signs sensor. After a defined number of compressions, for example 30 compressions, the helper is requested to give the person respiration. The defined number of compressions can be stored in the data storage device.

According to a further advantageous embodiment of the invention, the flow tube is equipped with a connection to which a mouthpiece for the respiration of a person in an emergency situation can be connected. This connection is located preferably at the second end of the flow tube.

According to a further advantageous embodiment of the invention, the device is equipped with an energy storage device. The energy storage device supplies the processor, the output device and the flow sensor or the vital signs sensor with electricity.

According to a further advantageous embodiment of the invention, the device exhibits a charging station to which the energy storage device can be detachably connected. If the energy storage device is connected to the charging station, the energy storage device is charged.

According to a further advantageous embodiment of the invention, the device exhibits a readout station to which the data storage device is detachably connected. If the data storage device is connected to the readout station, the data stored in the data storage device is output to the readout station. For that purpose the readout station is equally equipped with a memory device.

According to a further advantageous embodiment of the invention, the device is equipped with a machine-readable marking on one outer side. A device can be identified by this marking and distinguished from other devices.

According to a further advantageous embodiment of the invention, the charging station or the readout station is equipped with a reader that reads the machine-readable marking on the outside of the device.

The method according to the invention with the features of claim 22 is characterized in that with the help of the device after a respiratory mask has been fitted to a person in a medical emergency situation, the gases exhaled by the person that pass through the flow tube are determined by a flow sensor and/or a vital signs sensor arranged in the flow tube. The flow sensor determines the mass or volumetric flow of the gas flowing in the flow tube. The vital signs sensor determines the proportion of oxygen and carbon dioxide in the air flowing in the flow tube. The mass and volumetric flow and the proportion of oxygen and carbon dioxide depend on the penetration depth. Within a specified range, the mass or volumetric flow of the air flowing out of the person rises as the penetration depth in the cardiac massage is increased. Furthermore, the proportion of oxygen and carbon dioxide changes in a characteristic way as the penetration depth in the cardiac massage is increased. The processor processes the mass or volumetric flow and/or the proportion of oxygen and carbon dioxide into a characteristic output signal for the penetration depth, using the circumstances stated. This output signal is referred to as the penetration depth signal. The output signal is output by the output device.

The helper receives preferably not merely general advice for the cardiac massage, but specific instructions that are adapted to the condition of the person in an emergency situation. Determination of the mass and volumetric flow and of the proportion of oxygen and carbon dioxide, along with output of the output signal, continues while first aid is being administered. As a result, the instructions given to the helper during the course of first aid can be adapted continually to any changes in the condition of the person in an emergency situation.

According to a further advantageous embodiment of the invention, the gases flowing in the flow tube in both directions are determined by the flow sensor and/or by the vital signs sensor. This means that not only is the mass or volumetric flow and/or the proportion of oxygen and carbon dioxide in the air flowing out of the person detected, but also the breathing air fed to the patient.

According to a further advantageous embodiment of the method according to the invention, the output device indicates whether the mass or volumetric flow determined and/or the proportion of oxygen and carbon dioxide determined or the output signal derived therefrom or another parameter derived therefrom lies within a specified range for resuscitation. The range is specified such that a circulation of the blood that provides a blood supply to the brain takes place within this range.

According to a further advantageous embodiment of the method according to the invention, before a cardiac massage starts the mass or volumetric flow and/or the proportion of oxygen and carbon dioxide in the air flowing out of the person's nose and/or mouth is determined by means of the flow sensor. Whether the mass or volumetric flow determined and/or the proportion of oxygen and carbon dioxide is within a specified range for spontaneous breathing is output by the output device. In this way it can be established whether the person is breathing independently.

According to a further advantageous embodiment of the invention, the mass or volumetric flow determined and/or the proportion of oxygen and carbon dioxide determined is compared by the processor with specified threshold values for respiration. Based on the comparison, a respiration output signal is generated, indicating whether a person in a medical emergency situation requires respiration. The respiration output signal is output by the output device. The respiration output signal can for example be generated after a defined number of compressions, where the number of compressions is set on the basis of the mass or volumetric flow determined or on the basis of the proportion of oxygen and carbon dioxide determined. In addition, the respiration output signal can also be generated from the measured values for the mass or volumetric flow or the proportion of oxygen and carbon or from the temporal changes in the measured values.

According to a further advantageous embodiment of the invention, the acceleration with which the thorax is compressed is determined by means of an acceleration sensor during compression of the thorax. A characteristic output signal for the penetration depth is generated from the acceleration determined. The output signal is output as an output signal by means of the output device. The output device indicates whether the acceleration determined or the output signal derived from it or another parameter derived from the acceleration lies within a specified range for resuscitation.

According to a further advantageous embodiment of the invention, the output signal is output not only to the helper, but also to third parties who are involved in the subsequent medical care of the person in an emergency situation. These parties will typically be medical specialists such as paramedics, nurses or doctors, rather than first responders.

Further advantages and advantageous embodiments of the invention can be obtained from the following description, the drawing and the claims.

### DRAWING

Model embodiments of the invention are represented in the drawing. Illustrations:

FIG. 1 First model embodiment of a device for monitoring compressions in a cardiac massage in side view,

FIG. 2 Device according to FIG. 1 separated into its individual components,

FIG. 3 Flow tube with push-on part of the device according to FIG. 1 in a perspective view,

FIG. 4 Flow tube with push-on part according to FIG. 3 in a view from below,

FIG. 5 Flow tube according to FIG. 3 with push-on part lifted off,

FIG. 6 Flow tube according to FIG. 3 in an exploded view,

FIG. 7 Detail of FIG. 6 showing the sensor, the sensor circuit board and the sensor sleeves,

FIG. 8 Push-on part according to FIG. 3,

FIG. 9 Second model embodiment of a device for monitoring compressions in a cardiac massage in a view from the front,

FIG. 10 Device according to FIG. 9 in a side view,

FIG. 11 Flow tube and push-on part of the device according to FIG. 9 in a perspective view,

FIG. 12 Flow tube of the device according to FIG. 9 in a perspective view,

FIG. 13 Push-on part of the device according to FIG. 9 in a perspective view from above,

FIG. 14 Push-on part of the device according to FIG. 9 in a perspective view from below,

FIG. 15 Push-on part of the device according to FIG. 9 arranged on the arm of a helper,

FIG. 16 Third model embodiment of a device for monitoring compressions in a cardiac massage in a perspective view from above,

FIG. 17 First model embodiment of a charging and readout station for a device for monitoring compressions in a cardiac massage according to FIG. 16 in a perspective view from above,

FIG. 18 Second model embodiment of a charging and readout station for a device for monitoring compressions in a cardiac massage according to FIG. 16 in a perspective view from above.

### DESCRIPTION OF THE MODEL EMBODIMENTS

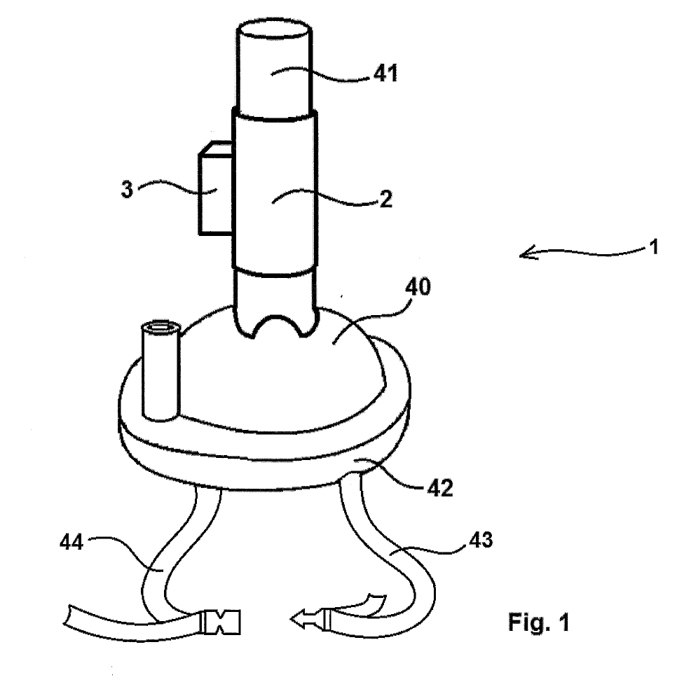

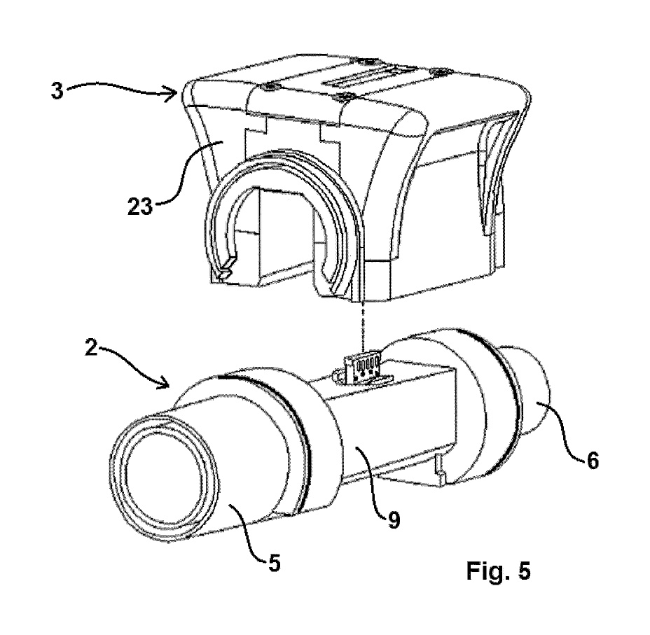

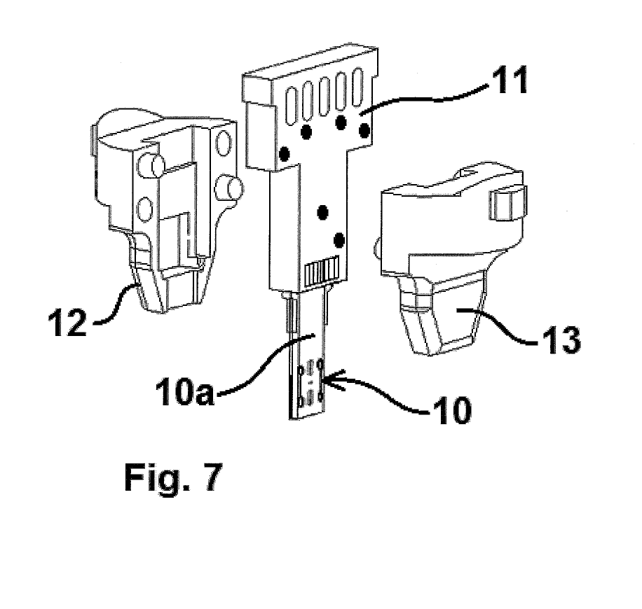

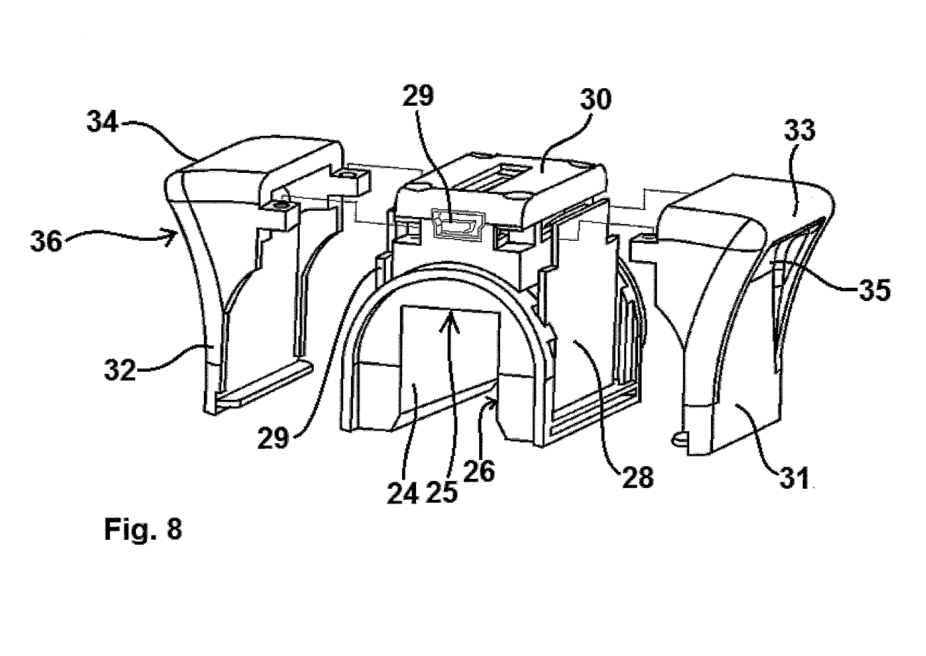

FIGS. 1 to 8 show a first model embodiment of a device for monitoring compressions in a cardiac massage. The device exhibits a flow tube 2 and a push-on part 3. A respiratory mask 40 can be connected to a first end and a mouthpiece 41 to a second end of the flow tube 2. The respiratory mask 40 is equipped with an elastic edge 42 so that it forms an airtight seal on the face of the casualty or person in an emergency situation. The respiratory mask 40 is usually pressed onto the face of a person with a degree of pressure so that no air can escape under the edge. To fasten the respiratory mask to a person, there are straps 43, 44 arranged at two points on the respiratory mask. The two straps 43, 44 are placed around the head of a person and connected. As a result, the respiratory mask and, with it, the entire device, is secured to a person.

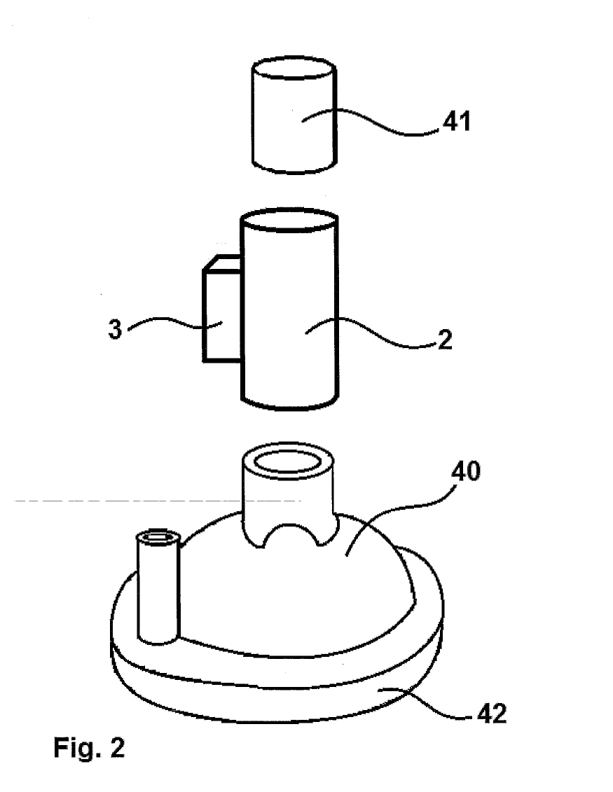

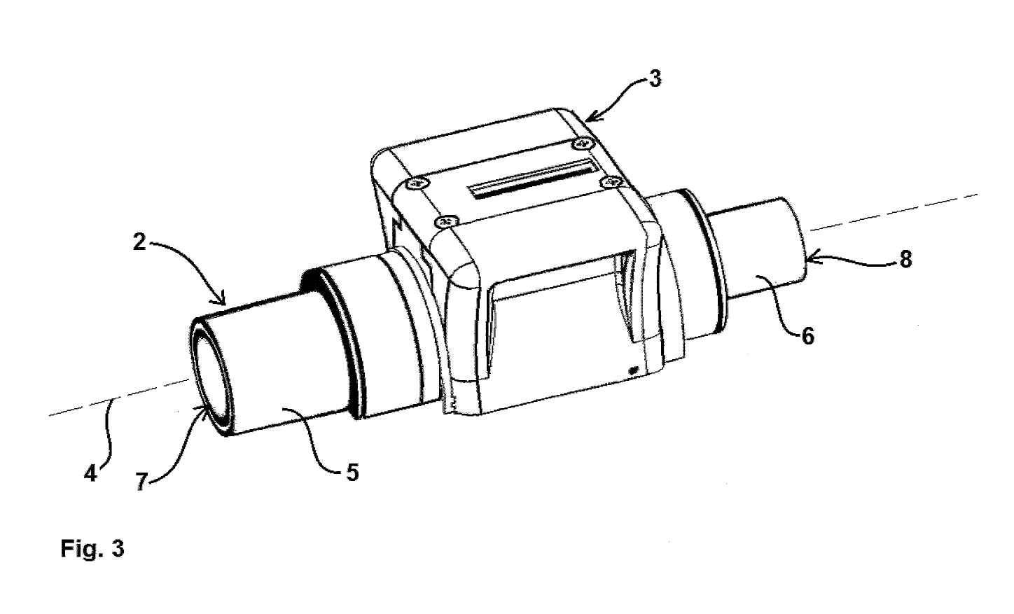

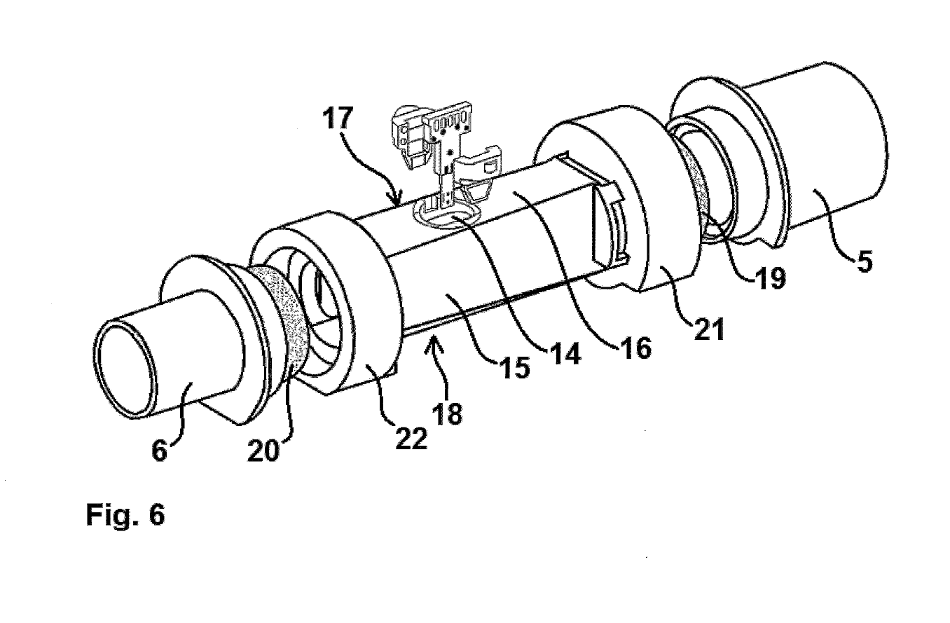

FIGS. 3 to 8 represent the flow tube 2 with the push-on part 3 shown in FIGS. 1 and 2. The flow tube is an elongate hollow body. Its longitudinal axis 4 corresponds to the direction of flow of the air flowing through the flow tube. The flow tube 2 exhibits a first end 5 and a second end 6. Both ends take the form of a cylindrical end section with a circular cross-sectional area. With these end sections, one end of the flow tube 2 is introduced into the respiratory mask 40 and the other end into the mouthpiece 41. The flow tube is open at the first face end 7 and at the second face end 8. Other than that, the flow tube is essentially closed. The flow channel of the flow tube runs between the first end 5 and the second end 6, each with a circular cross-section. The air flowing in and out through the two ends 5, 6 flows through this. There is a sensor section 9 of the flow tube 2 in the flow channel. A flow sensor 10 is arranged on the sensor section 9. The attachment of the sensor 10 is discernible in FIG. 6. The flow sensor 10 is equipped with a sensor fixture device 10a and a sensor circuit board 11. As well as the flow sensor 10, further sensors, in particular a vital signs sensor, can be arranged on the sensor fixture device 10a. The sensor circuit board 11 forms an extension of the sensor fixture device 10a. The sensor fixture device 10a can also be part of the sensor circuit board 11. The sensor circuit board 11 is held by two sensor sleeves 12 and 13. The sensor sleeves are adapted to a sensor opening 14 in the sensor section 9. The sensor circuit board 11 is pressed into the sensor opening 14 with the help of the sensor sleeves 12 and 13, thus locating the flow sensor. This clamps the sensor circuit board 11 between the two sensor sleeves 12, 13 and seals the sensor opening 14. The sensor now extends into the flow channel of the flow tube 2.

The sensor section 9 is limited by four side walls 15, 16, 17, 18. The two side walls 15 and 17 are flat and run at an angle of 5° to each other. The angle can also lie between 2° and 20°. The side wall 16 is likewise flat. It is adjacent to the two side walls 15 and 17. The fourth side wall 18 can likewise be flat or convex. If the fourth side wall 18 is flat in design, it is preferably parallel to the side wall 16. The four side walls 15, 16, 17, 18 produce the form of a truncated pyramid. The cross-section of the sensor section 9 is thus smaller at the end facing the cylindrical end section 5 of the flow tube 2 than at the end facing the cylindrical end section 6 of the flow tube 2. This is especially discernible in FIG. 2. Between the two ends, the cross-section decreases continuously perpendicular to the longitudinal axis of the flow tube 2. The cross-section increases continuously in the opposite direction.

A first filter 19 and a second filter 20 are discernible in FIG. 6. They are arranged on the ends of the two end sections of the flow tube 2 facing the sensor section 9 that are assigned to the first end 5 and the second end 6. The sensor section 9 is equipped at its two ends with sleeves 21, 22 that encompass the cylindrical end sections 5, 6.

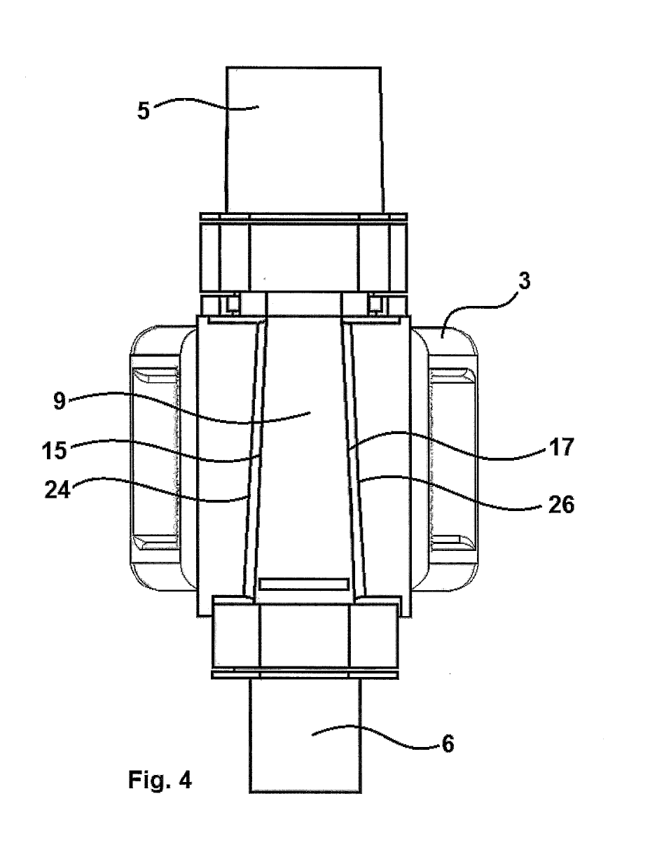

The push-on part 3 exhibits a housing 23. The housing 23 has a U-shape. It encompasses the sensor section 9 of the flow tube 2 from three sides so that the flat side walls 15, 16 and 17 are entirely covered by the push-on part. To that end, the push-on part exhibits three corresponding, flat side walls 24, 25 and 26 that rest on the side walls 15, 16 and 17. This is discernible in FIG. 4 with regard to the side walls 24 and 26 of the push-on part 3 and with regard to the side walls 15 and 17 of the sensor section 9. The two opposing side walls 24 and 26 of the push-on part 3 create a clamping force with which the push-on part 3 is connected to the flow tube 2 when it is pushed on.

In the housing 23 of the push-on part 3 there are circuit boards 27 and 28 on which electrical and electronic components are arranged. These are in particular a memory device and a processor. In addition an analog-to-digital converter and a DC voltage source can be arranged on the circuit boards. A connector socket not discernible in the drawing and into which the upper end of the circuit board 11 of the sensor 10 is inserted is arranged on the side wall 25. The connector socket is connected to the circuit boards 27, 28. It forms the interface to the sensor 10. In addition a connector socket 29 that forms an interface with an external reader—not represented in the drawing—is arranged on the front side of the housing 23. A display device can in addition be integrated into a cover 30 of the housing 23.

The two outer housing sections 31 and 32 cover the circuit boards 27, 28. They in addition exhibit handle elements 33, 34 that protrude outwards. These are equipped with grip recesses 35, 36 on their underside. The handles 33, 34 and the handle recesses 35, 36 facilitate pushing the push-on part 3 onto the flow tube 2 and lifting push-on part 3 off the flow tube 2.

To monitor compressions in a cardiac massage, the respiratory mask 40 is fitted over the nose and mouth of a person in a medical emergency situation and secured to the person's head with the straps 43, 44. The sensor is either already activated or is specifically activated. It determines the gases flowing through the flow tube 2. These gases are analyzed by the processor to obtain an output signal. This is output by an output device. The determination of the parameters for the gases flowing in the flow tube also continues during first aid. As a result, the instructions given to the helper during the course of first aid can be adapted continually to any changes in the condition of the person in an emergency situation.

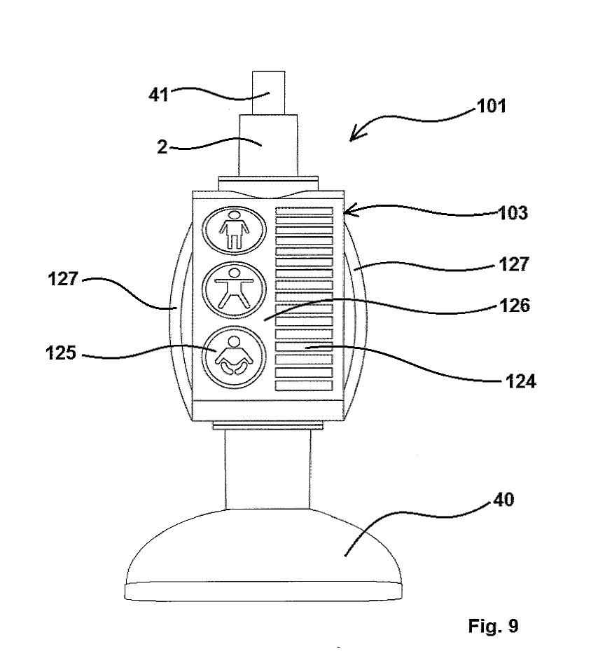

FIGS. 9 to 15 show a second model embodiment of a device 101 for monitoring compressions in a cardiac massage. The device essentially corresponds to the first model embodiment with regard to the flow tube 2. In addition, a respiratory mask 40 and a mouthpiece 41 can be fitted on the flow tube of the device 101 analogously to the first model embodiment. The reference numbers for these components therefore correspond to those in the first model embodiment. The second model embodiment differs from the first model embodiment with regard to the push-on part 103. In a housing 123 of the push-on part 103 are arranged a processor not discernible in the drawing and an output device 124. They form an optical display device with several elongate light elements arranged parallel one above the other. The individual light elements exhibit light-emitting diodes in different colors. If only the bottom light element is switched on, no mass or volumetric flow is determined in the flow tube. If only the top light element is switched on, a maximum mass or volumetric flow is determined. The top and bottom light elements have different colors. The light elements between them have a third color. They indicate a mass or volumetric flow between the minimum and the maximum. The symbols on the left next to the light elements are arranged on buttons or pushbuttons. These are part of an input device 125. The symbols stand for adult, child or infant. By operating a button, the helper selects the group of people to which the person in a medical emergency situation belongs. The maximum and minimum of the mass or volumetric flow of the air flowing out of the person differs between these three groups of people.

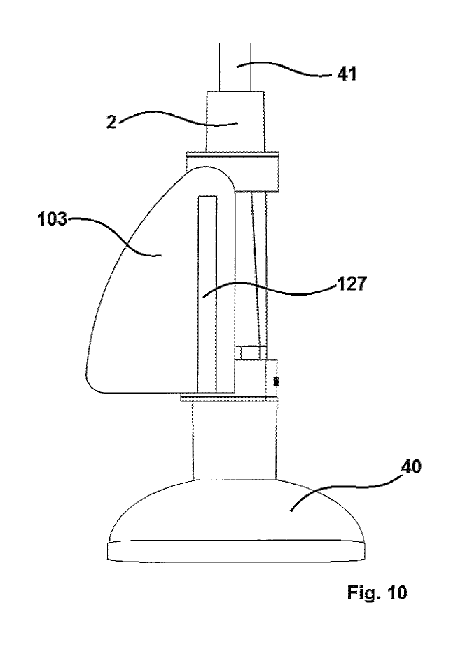

The surface of the push-on part 103 facing the viewer in FIG. 9 is an optically active surface 126. The light elements of the output device 124 are integrated into this surface 126. This surface is convex. This is discernible in the side view according to FIG. 10.

The push-on part 103 exhibits two handles 127 at the side on the housing 123. The push-on part 103 can be removed from the flow tube 2 with the help of these handles.

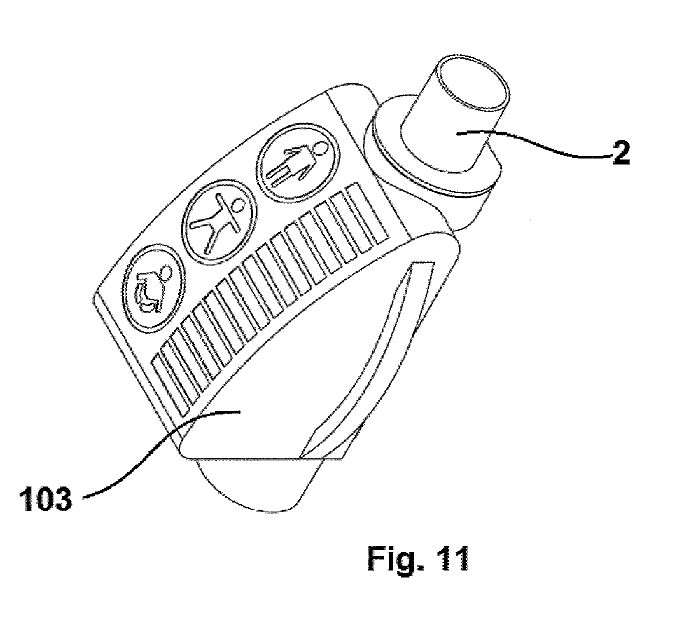

FIG. 11 shows the flow tube 2 and the push-on part 103 in a perspective view.

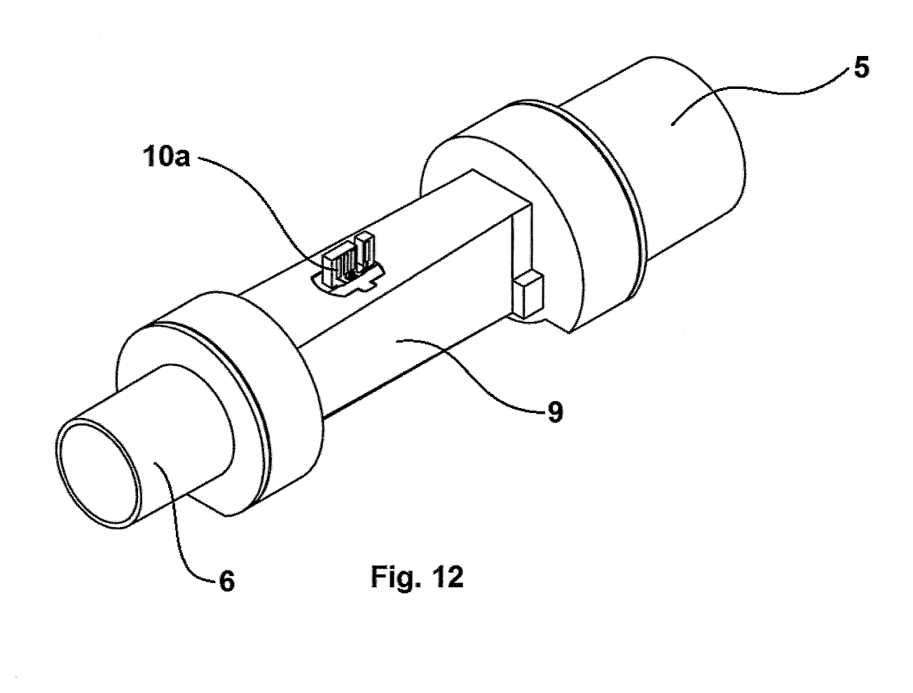

FIG. 12 shows the flow tube 2 with the two ends 5 and 6 and the sensor section 9. With regard to the remaining components of the flow tube, reference is made to the above description for the first model embodiment.

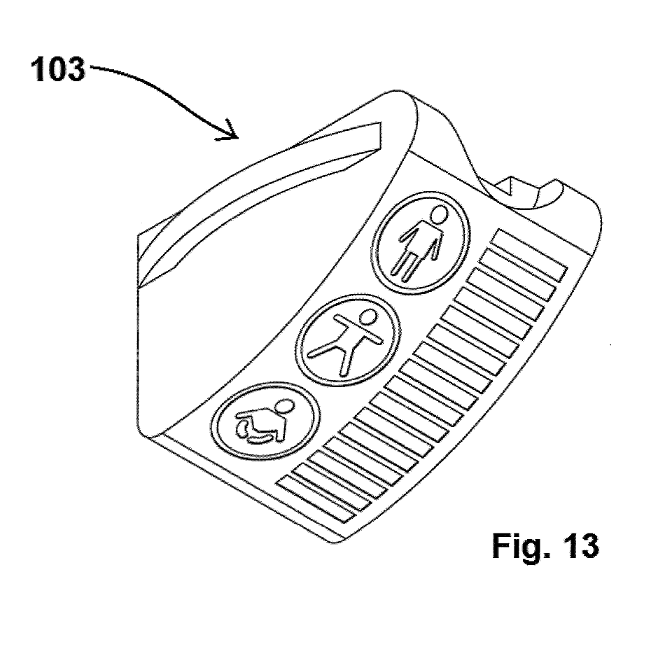

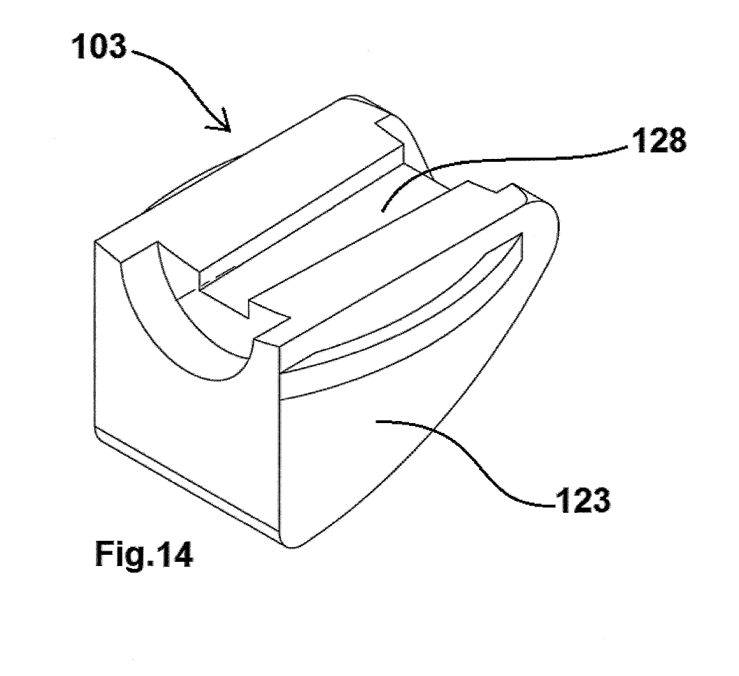

The push-on part 103 is represented in the FIGS. 13 and 14. The recess 128 in the housing 123 in which the flow tube is inserted is discernible in FIG. 14.

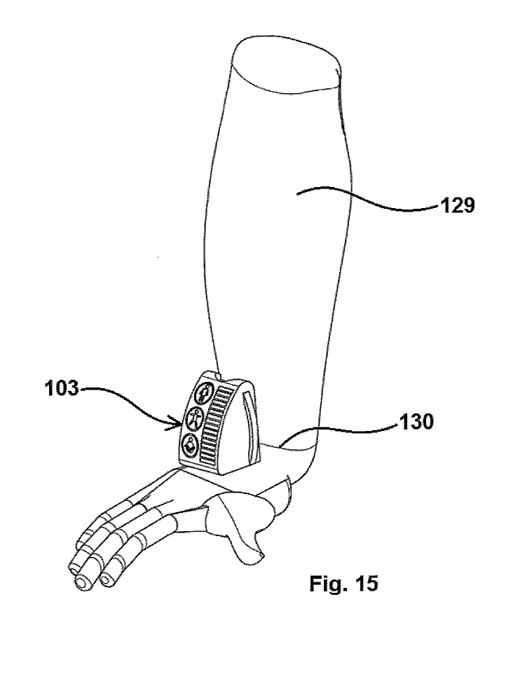

FIG. 15 shows the push-on part 103 that is arranged on an arm 129 of a helper. To that end, the push-on part 103 is fastened to the arm with an arm fastening device 130 in the form of a strap. An acceleration sensor not discernible in the drawing is arranged in the push-on part.

To monitor compressions in a cardiac massage of a person, the device 101 is equipped with a respiratory mask 40. This is fitted over the mouth and nose of the person. Using the buttons 125, it is selected which group of people the person belongs to. The flow sensor in the flow tube determines the mass or volumetric flow of the air flowing out of the person. The mass or volumetric flow determined is displayed with the output device 124. If the mass or volumetric flow is too low, the helper administers a cardiac massage. If necessary, air is also supplied via the mouthpiece. In the cardiac massage, the mass or volumetric flow of the air flowing out of the person is determined. If no mass or volumetric flow is identified even though the penetration depth is increased, this is a sign of obstructed airways. In this case the helper can remove the push-on part 103 from the flow tube 2 and fasten it to his/her arm with the help of the strap 130. The penetration depth is now determined with the help of the acceleration sensor integrated into the housing 123. If the airways are not obstructed, the mass or volumetric flow of the air flowing out of the person can be continuously detected with every compression of the person's thorax, an output signal generated and displayed on the output device 124. The mass or volumetric flow is a measure of the penetration depth in the cardiac massage. The helper learns from the display device whether the penetration depth was too low, sufficient or too high. He/she can modify the penetration depth accordingly in the next compression.

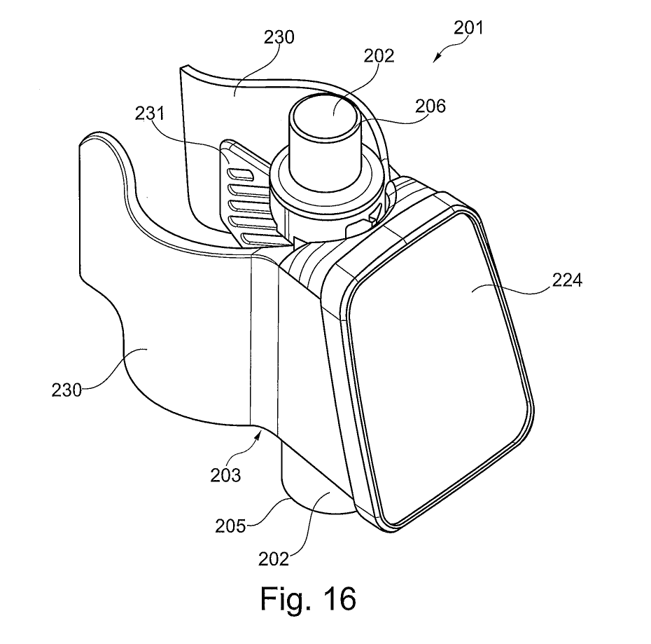

FIG. 16 represents a third model embodiment of a device 201 for monitoring compressions in a cardiac massage. The device exhibits a flow tube 202 and a push-on part 203 which are connected to each other detachably by a plug-in connection. The flow tube 202 essentially corresponds to the flow tube 2 of the first and second model embodiment. It exhibits a first end 205, a second end 206 and a continuous flow channel for air from the first to the second end. The entry into the flow channel on the second end 206 is discernible in the drawing. A flow sensor and a vital signs sensor, which are not discernible in the drawing, are arranged in the flow channel. Their composition and arrangement correspond to the representation in FIG. 6.

Unlike the flow tube 2, the flow tube 202 is equipped with a handle part 231. This facilitates detaching the flow tube 202 from the push-on part 203.

A respiratory mask can be fastened to the first end 205 of the flow tube 202. A mouthpiece for respiration can be fastened to the second end 206 of the flow tube 206. Respiratory mask and mouthpiece are not represented in FIG. 16. A respiratory mask 40 and a mouthpiece 41 according to FIG. 1 can be connected to the device according to FIG. 16.

The third model embodiment of the device differs from the first and second model embodiment with regard to the push-on part 203. In a housing 223 of the push-on part 203 are arranged a processor not discernible in the drawing, an output device 224 and an acceleration sensor not discernible in the drawing. The output device 224 is an optical display device in the form of a screen. The screen can also take the form of a touchscreen, so that it can be used for the input and output of data.

The push-on part 203 exhibits an arm bangle 230 with which it can be fastened to the arm of a helper after the flow tube has been detached.

The functioning principle of the device according to the third model embodiment is analogous to the functioning principle of the second model embodiment. In this regard, reference is made to the above description of the second model embodiment.

An energy storage device, for example a rechargeable battery, and a data storage device that are not represented in the drawing are arranged in the push-on part.

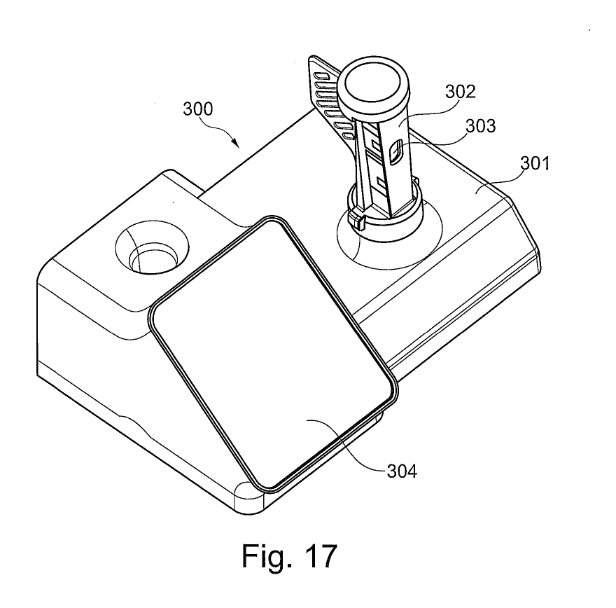

FIG. 17 represents a first model embodiment of a charging and readout station 300 for a device for monitoring compressions in the cardiac massage. The charging and readout station exhibits a housing 301, a coupling part 302 with a connector socket 303 and an output device 304 in the form of a screen. The outer form of the coupling part 302 matches the form of the flow tube 202 according to FIG. 16. The push-on part 203 that is represented in FIG. 16 can be pushed onto the coupling part 302 after the flow tube 202 has been removed from the push-on part 203. An electrical contact between the push-on part 203 and the coupling part 302 is established by a connector socket 303. The push-on part 203 is equipped with a plug not represented in the drawing, on the side of the housing 223 facing away from the screen 224. Alternatively a connector socket can also be provided on the push-on part 203 and a plug provided on the coupling part 302.

If the push-on part 203 according to FIG. 16 is fitted on the coupling part 302 according to FIG. 17, an energy storage device arranged in the push-on part and not represented in the drawing is charged in the push-in part 203. In addition, the data stored in a data storage device of the push-on part 203 is transferred to a memory device of the charging and readout station 300. The data storage device of the push-on part 203 and the memory device of the charging and readout station 300 are not represented in the drawing.

A user is informed of the status of the energy storage device charging process and of readout of data via the screen of the output device 304. In addition, the user can be informed of particularities of the device for monitoring compressions in the cardiac massage via the output device.

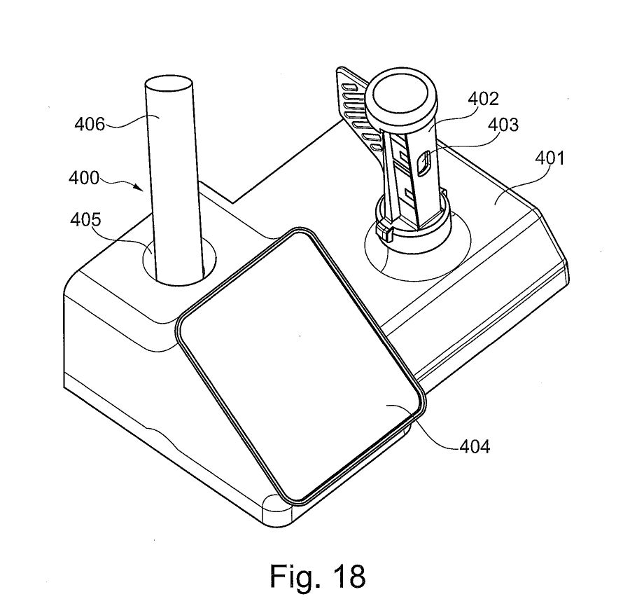

FIG. 18 represents a second model embodiment of a charging and readout station 400 for a device for monitoring compressions in the cardiac massage. The charging and readout station 400 essentially corresponds to the charging and readout station 300. It exhibits a housing 401, a coupling part 402 with a connector socket 403 and an output device 404 in the form of a screen. Reference is made to the above description for FIG. 17 with regard to the functioning principle. Furthermore, the charging and readout station 400 is equipped with a stylus 406 with which the machine-readable markings on the outside of a push-on part 203 can be read. Such a marking is not discernible in the drawing. The marking may be for example a barcode, and the stylus 406 a barcode reader. The stylus is accommodated detachably in a recess 405 of the housing 401.

All features of the invention can be material to the invention both individually and in any combination.

### REFERENCE NUMBERS

1 Device for monitoring compressions in a cardiac massage
2 Flow tube
3 Push-on part
4 Longitudinal axis
5 First end of the flow tube
6 Second end of the flow tube
7 First face end of the flow tube
8 Second face end of the flow tube
9 Sensor section
10 Sensor
10a Sensor fixture device
11 Sensor circuit board
12 Sensor sleeve
13 Sensor sleeve
14 Sensor opening
15 Side wall
16 Side wall
17 Side wall
18 Side wall
19 First filter
20 Second filter
21 Sleeve
22 Sleeve
23 Housing
24 Side wall
25 Side wall
26 Side wall
27 Circuit board
28 Circuit board
29 Connector socket
30 Cover
31 Outer housing part
32 Outer housing part
33 Handle element
34 Handle element
35 Grip recess
36 Grip recess
37
38
39
40 Respiratory mask
41 Mouthpiece
42 Elastic edge
43 Strap
44 Strap
101 Device for monitoring compressions in a cardiac massage
103 Push-on part
123 Housing
124 Display device
125 Input device
126 Surface
127 Handle element
128 Recess
129 Arm
130 Arm fastening device
201 Device for monitoring compressions in a cardiac massage
202 Flow tube
203 Push-on part
205 First end of the flow tube
206 Second end of the flow tube
223 Housing
224 Output device
230 Arm bangle
231 Handle part
300 Charging and readout station
301 Housing
302 Coupling part
303 Connector socket
304 Output device
400 Charging and readout station
401 Housing
402 Coupling part
403 Connector socket
404 Output device
405 Recess
406 Stylus

1. Device for monitoring compressions in a cardiac massage of a person in a medical emergency situation, with a flow tube (2, 202), with a first end (5, 205) and a second end (6, 206), wherein the first end (5, 205) is connectable to a respiratory mask (40) that fits over the nose and mouth of a person in such a way that air flows out of the respiratory mask (40) into the flow tube (2, 202) and out of the flow tube (2, 202) into the respiratory mask (40),
with a flow channel on the flow tube (2, 202) for a continuous passage of air from the first end (5, 205) to the second end (6, 206),
with at least one flow sensor (10) for determining a mass or volumetric flow arranged in the flow channel of the flow tube (2, 202), by means of which a mass or volumetric flow of the air flowing out of the nose and/or mouth of the person and through the flow channel during a cardiac massage as a result of compression of the thorax can be determined to a penetration depth,
and/or with a vital signs sensor arranged on the flow tube, by means of which a proportion of oxygen and carbon dioxide in the air flowing out of the nose and/or mouth of the person and through the flow channel during a cardiac massage as a result of compression of the thorax can be determined to a penetration depth,
with a processor that generates a characteristic output signal for the penetration depth from the mass or volumetric flow determined and/or from the proportion of oxygen or carbon dioxide determined,
with an output device (124, 224) that issues the output signal.

2. Device according to claim 1, wherein the output device (124, 224) is designed such that it indicates whether the mass or volumetric flow determined or the proportion of oxygen and carbon dioxide determined or the output signal derived from this or another parameter derived from the mass or volumetric flow or the proportion of oxygen and carbon dioxide lies within a specified range for resuscitation.

3. Device according to claim 1, wherein the output device (124, 224) exhibits an optical display device.

4. Device according to claim 3, wherein the optical display device (124) has several light sources.

5. Device according to claim 3, wherein the optical display device (224) has a screen.

6. Device according to claim 3, wherein the optical display device exhibits an optically active surface (126) on which the output signal is displayed, wherein the optically active surface (126) is a convex surface or essentially a flat surface, and wherein the flat optically active surface includes an angle of more than 0° and less than 90° with the longitudinal direction of the flow tube (2), where the longitudinal direction essentially corresponds to the direction of flow of the gases flowing through the flow tube (2).

7. Device according to claim 1, wherein the output device has an acoustic display device.

8. Device according to claim 1, wherein it is equipped with an interface over which the data can be output to an external device for emergency care, to a medical device, to a computer, to a mobile communications device and/or to a readout station.

9. Device according to claim 1, wherein a filter (19) is arranged in the flow channel between the first end (5, 205) and the second end (6, 206) of the flow tube (2, 202).

10. Device according to claim 1, wherein it is equipped with a push-on part (3, 103, 203) that can be connected detachably to the flow tube (2, 202), and wherein the processor and the output device (29, 124, 224) are arranged in the push-on part (3, 103, 203).

11. Device according to claim 10, wherein the push-on part (103, 203) can be laid on the thorax of the person or fastened to the arm of an assisting person, wherein the push-on part is equipped with an acceleration sensor with which the acceleration at which the thorax of the person receiving cardiac massage is compressed can be measured.

12. Device according to claim 11, wherein the processor is designed such that it generates an output signal from the acceleration measured by the acceleration sensor, and wherein the output device is designed such that it issues this output signal.

13. Device according to claim 12, wherein the output device (124, 224) is designed such that it indicates whether the acceleration determined or the output signal derived from it or another parameter derived from the acceleration lies within a specified range for resuscitation.

14. Device according to claim 1, wherein the processor is designed such that it generates a respiration output signal if the mass or volumetric flow determined and/or the proportion of oxygen or carbon dioxide determined passes above or below a specified threshold value for respiration, and wherein the output device (124, 224) is designed such that it issues the respiration output signal.

15. Device according to claim 1, wherein the flow tube (2, 202) is equipped with a connection to which a mouthpiece (41) for the respiration of a person in an emergency situation can be connected.

16. Device according to claim 1, wherein it is equipped with an energy storage device.

17. Device according to claim 16, wherein it is equipped with a charging station (300, 400) to which the energy storage device can be connected detachably, and which charges the energy storage device.

18. Device according to claim 1, wherein it is equipped with a data storage device which stores the mass or volumetric flow determined with the flow sensor and/or the proportion of oxygen or carbon determined with the vital signs sensor.

19. Device according to claim 18, wherein it is equipped with a readout station (300, 400) to which the data storage device can be connected detachably, and which reads out the data stored in the memory device.

20. Device according to claim 1, wherein it is equipped with a machine-readable marking on one outer side.

21. Device according to claim 17, further comprising a machine-readable marking on one outer side, and wherein the charging station (400) or the readout station (400) is equipped with a reader (406) that reads the machine-readable marking.

22. Method of monitoring compressions in a cardiac massage of a person in a medical emergency situation with the help of a respiratory mask that fits over the nose and mouth of the person and with a device that comprises a flow tube (2, 202) that can be connected to the respiratory mask with a continuous flow channel that opens out into the respiratory mask (40) at one end (5, 205) and exhibits a through opening at a second end (6, 206), a flow sensor with which a mass or volumetric flow of an air flowing in the flow tube can be determined, and/or a vital signs sensor with which a proportion of oxygen and carbon dioxide in an air flowing in the flow tube can be determined, a processor and an output device (124, 224) comprising the following process steps:
fitting the respiratory mask (40) over a nose and mouth area of the person,
determining a mass or volumetric flow of the air flowing out of the nose and/or mouth of the person and through the flow tube (2, 202) in a cardiac massage as a result of compression of the thorax to a penetration depth,
and/or determining a proportion of oxygen and carbon dioxide in the air flowing out of nose and/or mouth of the person and through the flow tube (2, 202) in a cardiac massage as a result of compression of the thorax to a penetration depth, generating a characteristic output signal for the penetration depth from the mass or volumetric flow determined and/or the proportion of oxygen and carbon dioxide,
output of the output signal by means of the output device (124, 224).

23. Method according to claim 22, wherein it is indicated by the output device (124, 224) whether the mass or volumetric flow determined and/or the proportion of oxygen and carbon dioxide determined or the output signal derived from this or another parameter derived from the mass or volumetric flow or the proportion of oxygen and carbon lies within a specified range for resuscitation.

24. Method according to claim 22, wherein the mass or volumetric flow of the air flowing out of the nose and/or mouth of the person is determined by means of the flow sensor (10) before cardiac massage starts, and wherein it is output by the output device (124, 224) whether the mass or volumetric flow determined is within a specified range for spontaneous breathing.

25. Method according to claim 22, wherein the proportion of oxygen and carbon dioxide in the air flowing out of the nose and/or mouth of the person is determined by means of the vital signs sensor (10) before cardiac massage starts, and wherein it is output by the output device (124, 224) whether the proportion of oxygen and carbon dioxide determined is within a specified range for spontaneous breathing.

26. Method according to claim 22, wherein the mass or volumetric flow determined and/or the proportion of oxygen and carbon dioxide determined is compared with specified threshold values for respiration by means of the processor, and wherein the comparison is used to generate a respiration output signal that indicates whether a person in a medical emergency situation must be given respiration, and that wherein the respiration output signal is output with the output device (124, 224).

27. Method according to claim 22, wherein the acceleration with which the thorax is compressed is determined by means of an acceleration sensor during compression of the thorax, wherein a characteristic output signal for the penetration depth is generated from the acceleration determined, wherein the output signal is output by means of the output device (124, 224) and wherein it is indicated by the output device (124, 224) whether the acceleration determined or the output signal derived from it or another parameter derived from the acceleration lies within a specified range for resuscitation.
